(19) United States
(12) Patent Application Publication
(10) Pub. No.: US 2019/0388610 A1
(43) Pub. Date: Dec. 26, 2019

(54) SPRING POWERED AMBULATORY INFUSION APPARATUS

(71) Applicant: Seik OH, Trabuco Canyon, CA (US)

(72) Inventor: Seik OH, Trabuco Canyon, CA (US)

(21) Appl. No.: 16/016,270

(22) Filed: Jun. 22, 2018

(51) Int. Cl. A61M 5/145; A61M 5/20

(52) U.S. Cl. CPC A61M 5/1454; A61M 2005/2073; A61M 2005/202; A61M 5/2033

(57) ABSTRACT

A spring powered ambulatory infusion apparatus includes a syringe module and an actuator module. The syringe module includes a syringe barrel for containing infusion medicine and a piston for discharging the infusion medicine. The actuator module includes compression springs to move the piston when the springs expand. Each spring is bent to reduce its overall length, and have a U-shape in its compressed state and a J-shape in its expanded state. When each spring expands, one end of the spring moves to push the piston while the other end is stationary such that the shape thereof changes from the U-shape to the J-shape.

### BACKGROUND

### Field

The present disclosure relates to a spring powered ambulatory infusion apparatus.

### Discussion of Related Technology

In general, there are three types of ambulatory infusion pumps, elastomeric bladder type pumps, pneumatic pumps and spring powered pumps. The elastomeric bladder pump is relatively small and light-weighted. However, it is difficult for this type of infusion pump to provide a uniform flow rate. On the other hand, the spring powered pump is more advantageous for providing a generally linear flow rate. However, the size of the spring powered pump is generally bigger than the elastomeric bladder type pump. The foregoing discussion in this section is to provide general background information, and does not constitute an admission of prior art.

### SUMMARY

One aspect of the invention provide a spring actuated infusion pump, which may comprise: a syringe module configured to contain infusion medicine, wherein the syringe module comprises a syringe barrel and a piston, wherein the syringe barrel comprises a bottom and side walls extending from the bottom in a direction, wherein the syringe barrel further comprises an outlet for discharging infusion medicine, wherein the piston is disposed in the syringe barrel and movable in the direction, wherein the piston is configured to push infusion medicine contained in the syringe barrel toward the outlet; and an actuator module configured to engage with the syringe module, wherein the actuator module is further configured to push the piston in the direction, wherein the actuator module comprises a housing, two or more spring guide channels arranged side by side in the housing and two or more compression springs, each of which is retained in the corresponding spring guide channel, wherein each spring guide channel comprises an first end, a second end and a rounded portion between the first end and the second end, wherein each spring is compressible and configured to expand between a compressed state and an expanded state, wherein each spring comprises a fixed end, a movable end and a bent portion in conformation with the rounded portion of the corresponding guide channel, wherein the actuator comprises spring supports, each of which configured to block the second end of the corresponding spring guide channel such that the fixed end contacts and is stopped by the corresponding support, wherein the movable end is configured to move in the direction and further configured to push the piston when the spring expands, which causes the piston to move in the direction, wherein each spring guide channel is configured to retain the corresponding spring therein such that the fixed end, the movable end and the rounded portion of the corresponding spring overlap the piston in the compressed state and the expanded state when viewed in the direction while the fixed end and the movable end are located at opposite sides of the infusion apparatus, wherein the movable end of a first one of the springs and the movable end of a second one of the springs are located at opposite sides of the infusion apparatus when viewed in the direction.

In the foregoing apparatus, the entire portion of each spring overlaps the piston when viewed in the direction. The piston may have a generally rectangular shape when viewed in the direction, wherein the movable end of the first spring is configured to push the piston at a first corner of the piston, and the movable end of the second spring is configured to push the piston at a second corner of the piston that is diagonally opposite to the first corner. Each spring may have a U-shape in the compressed state, and each spring has a J-shape in the expanded state and while the movable end moves in the direction.

Still in the foregoing apparatus, the movable end of each spring may be located in the housing of the actuator module in the compressed state and located in the syringe barrel in the expanded state while the fixed end is located in the housing of the actuator in both the compressed state and the expanded state. Both the movable end and the fixed end of each spring may face the piston in the compressed state and in the expanded state. The bent portion of each spring in the compressed state may be partly deformed to extend linearly in the expanded state. Each spring may be formed of a single wire that is coiled and interconnects the fixed end and the movable end.

Yet in the foregoing apparatus, each channel may further include a first straight extension connected to the first end and a second straight extension connected to the second end, and the rounded portion of each channel interconnects the first and second straight extensions. The rounded portion may be semi-circular or arcuate. The actuator module may further comprise a locking mechanism configured to maintain the springs in the compressed state. In the expanded state of the spring, the piston may contact the bottom of the syringe barrel.

Further in the foregoing apparatus, the infusion apparatus may further comprise a pressure plate that contacts the movable end of each spring and is coupled to the piston to push the piston when the springs expand. The actuator module may further comprise two or more buckling-prevention guides extending from the pressure plate, wherein the movable end of each spring is inserted in the corresponding buckling-prevention guide that has a bending stiffness greater than that of the corresponding spring. Each buckling-prevention guide may comprise an end portion that is deformable and configured to deform in conformation with the rounded portion of the corresponding spring guide channel when the buckling-prevention guide is inserted in the corresponding guide channel. The actuator module may further comprise two or more posts extending from the pressure plate, wherein each post is inserted in the movable end of the corresponding spring, and each post has a bending stiffness greater than that of the corresponding spring. Each buckling-prevention guide may comprise telescopic pipes that receive the movable end of the spring, wherein each of the telescopic pipes is movable with respect to the rest of the telescopic pipes when the spring moves the piston in the direction.

Another aspect of the invention provide a method of infusing medicine to a patient, wherein the method may comprise: providing a syringe module containing infusion medicine therein, wherein the syringe module comprises a syringe barrel and a piston, wherein the syringe barrel comprises a bottom and side walls extending from the bottom in a direction, wherein the syringe barrel further comprises an outlet for discharging the medicine, wherein the piston is disposed in the syringe barrel and movable in the direction, wherein the piston is configured to push infusion medicine contained in the syringe barrel toward the outlet; providing an actuator module configured to push the piston in the direction when the actuator is engaged with the syringe module, wherein the actuator module comprises a housing, two or more spring guide channels arranged side by side in the housing and two or more compression springs, each of which is retained by the corresponding spring guide channel, wherein each spring guide channel comprises a first end, a second end and a rounded portion between the first end and the second end, wherein each spring is compressible and configured to expand between a compressed state and an expanded state, wherein each spring comprises a fixed end, a movable end and a bent portion in conformation with the rounded portion of the corresponding guide channel, wherein the actuator comprises spring supports, each of which configured to block the second end of the corresponding spring guide channel such that the fixed end contacts and is stopped by the corresponding spring support, wherein the movable end is configured to move in the direction and further configured to push the piston when the spring expands, which causes the piston to move in the direction; engaging the syringe module with the actuator module to form an infusion apparatus, wherein, in an engaged state of the syringe and the actuator module, each spring guide channel is configured to retain the corresponding spring such that the fixed end, the movable end and the rounded portion of the corresponding spring overlap the piston in the compressed state and the expanded state when viewed in the direction while the fixed end and the movable end are located at opposite sides of the infusion apparatus, wherein the movable end of a first one of the springs and the movable end of a second one of the springs are located at opposite sides of the infusion apparatus when viewed in the direction, wherein the springs are held in the compressed state; releasing the springs of the actuator module from the compressed state such that the springs expand and the movable ends of the springs push the piston, which causes the piston to move in the direction for discharging the infusion medicine through the outlet; upon completion of infusion, detaching the syringe module from the actuator module; and retracting the springs to the compressed state.

The foregoing method may further comprise engaging another syringe module containing infusion medicine with the actuator module for infusion in which the springs are held in the compressed state, and releasing the springs from the compresses state for infusion. In the foregoing method, each spring may have a U-shape in the compressed state before engaging, each spring becomes a J-shape after releasing and each spring becomes back to a U-shape after retracting.

### BRIEF DESCRIPTION OF THE DRAWINGS

FIG. 1 is a perspective view of an infusion apparatus, showing a state thereof ready for use.

FIG. 2 is a front view of the infusion apparatus shown in FIG. 1.

FIG. 3 is a top view of the infusion apparatus shown in FIG. 1.

FIG. 4 is a bottom view of the infusion apparatus shown in FIG. 1.

FIG. 5 is an exploded view of the infusion apparatus shown in FIG. 1.

FIGS. 6, 7 and 8 are sectional views of the infusion apparatus taken along line A-A in FIG. 3, in which FIG. 6 shows a state ready for use, FIG. 7 shows a state when infusion is being performed and FIG. 8 shows a state when infusion is completed.

FIG. 9 is a section view of the infusion apparatus taken along line B-B in FIG. 8 for showing locations of buckling-prevention structures and springs according to one embodiment.

FIG. 10 is a perspective view of an infusion apparatus, showing a state when infusion is completed.

FIG. 11 is a perspective cutaway view of the infusion apparatus shown in FIG. 9, in which springs are not shown.

FIGS. 12, 13 and 14 are sectional views of an infusion apparatus according to another embodiment, taken along line A-A in FIG. 3, in which FIG. 12 shows a state ready for use, FIG. 13 shows a state when infusion is being performed and FIG. 14 shows a state when infusion is completed.

FIGS. 15A-15C show various shapes of a spring used in an infusion apparatus according to one embodiment, in which FIG. 15A shows the spring in its unbent, compressed state and bent, compressed state shown in FIGS. 1 and 6 to compare its height, FIG. 15B shows the spring in its unbent, expanded state and its bent, expanded state shown in FIG. 8 to compare its height, and FIG. 15C shows the spring which is unbent and free of exterior force.

FIG. 16 is a graph showing changes of spring force changes of various springs that can be used in an infusion apparatus.

FIG. 17 is a graph for comparing infusion pressure changes of various infusion devices.

FIG. 18 is a perspective view of an infusion apparatus, showing a state thereof before an actuator and a syringe are engaged with each other, in which infusion fluid is filled in the syringe barrel.

FIG. 19 is a perspective view of an infusion apparatus, showing a state thereof before an actuator and a syringe are engaged with each other, in which infusion fluid is not filled in the syringe barrel.

FIG. 20 is a perspective view of the infusion apparatus, showing a state when infusion fluid is supplied into the syringe barrel.

### DETAILED DESCRIPTION OF EMBODIMENTS

Embodiments are described with reference to the accompanying drawings below. The terminology used in the description presented herein is not intended to be interpreted in any limited or restrictive manner, simply because it is being utilized in conjunction with a detailed description of certain specific embodiments of the invention.

### Spring Powered Infusion Apparatus

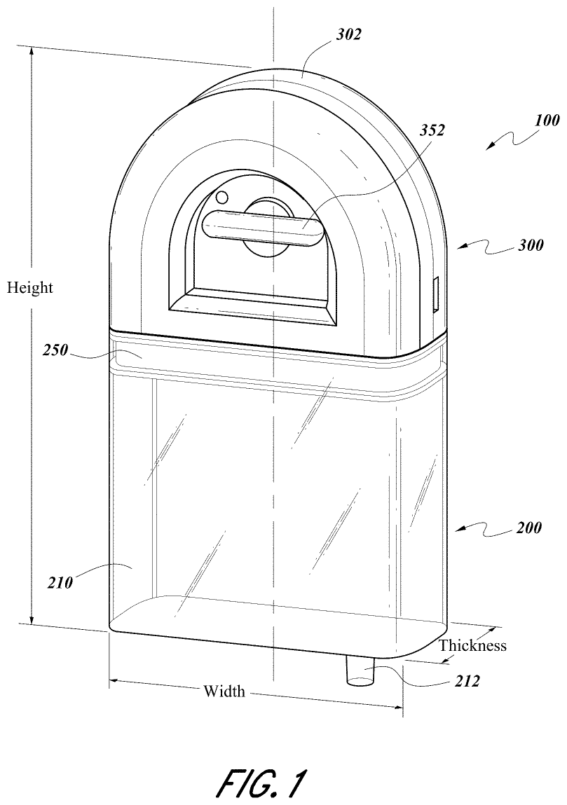

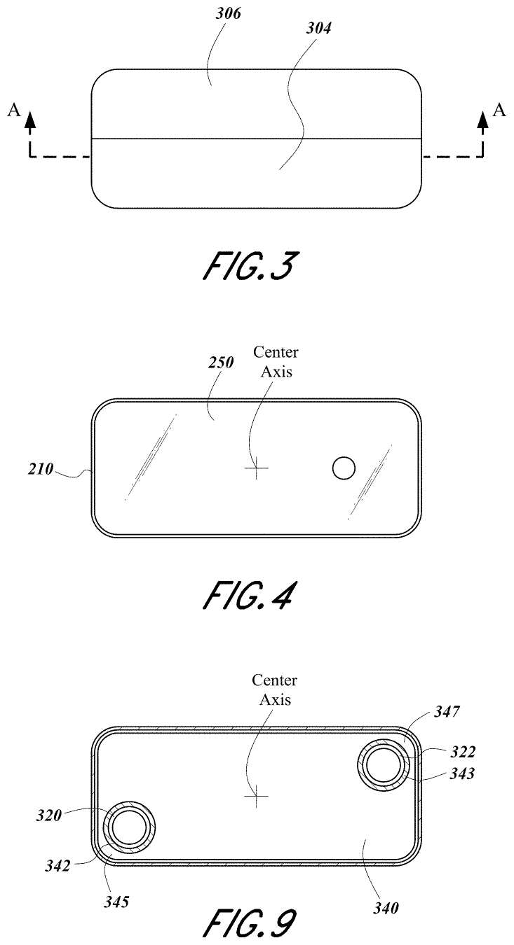

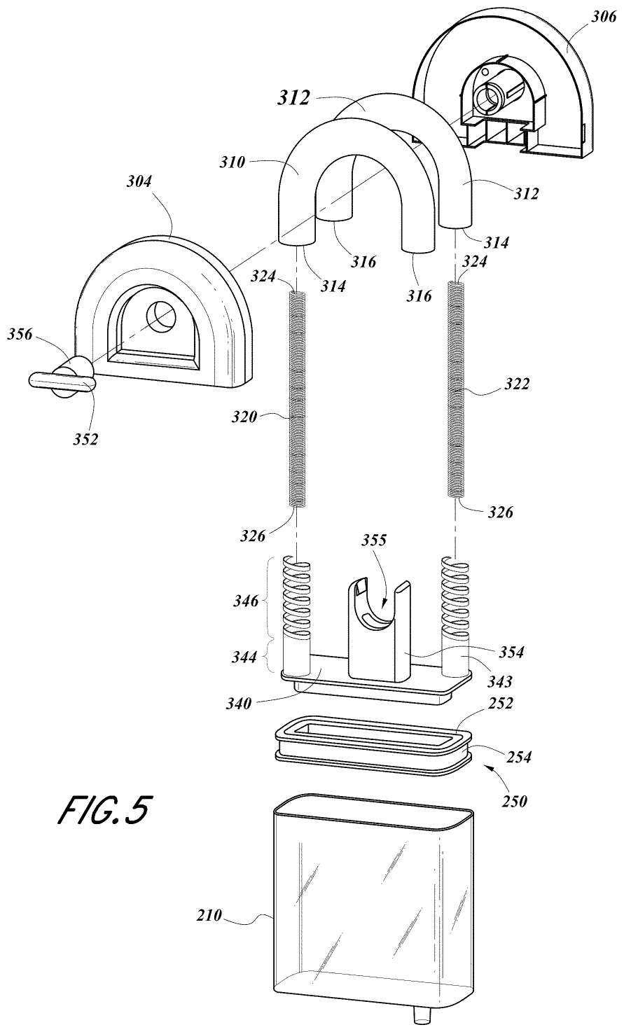

In embodiments, a spring powered infusion apparatus includes a syringe module and an actuator module. The syringe module includes a syringe barrel and a piston that is inserted and movable in the syringe barrel. The actuator module is attached to one end of the syringe module. The infusion apparatus has a height smaller than that of typical spring powered infusion pumps. In embodiments, the infusion apparatus has a generally rectangular sectional shape that has a width and a thickness as shown in FIGS. 1, 3 and 4.

### Springs of Infusion Apparatus

The actuator module includes compression springs to push the piston for discharging medicine fluid contained in the syringe barrel when the compression springs expand. Generally, it is advantageous to use a spring that has a less force decrease rate, a smaller spring constant, and a smaller ratio of compression displacement versus free length. Relatively long compression springs may provide the above characteristics. However, the longer the spring is, the bigger the size of the infusion apparatus (in particular, the height of the apparatus) is. The infusion apparatus according to embodiments of the present invention provides improved spring configuration for reducing the height of the apparatus while providing an accurate flow rate or a uniform flow rate.

### Bent Shape of Springs

In embodiments, the actuator module uses multiple compression springs (e.g., two springs) that are relatively long to provide an accurate flow rate. To reduce the height of the apparatus, however, each compression spring of the actuator module is bent to have a U-shape or J-shape.

### Side by Side Arrangement of Springs

Further, in the actuator module, two compression springs are not only bent but also arranged side by side such that the two springs can fit through or be accommodated in the rectangular sectional shape of the infusion apparatus having the width and the thickness. Thus, the spring powered infusion apparatus according to embodiments has a reduced size while not compromising advantages of providing accurate flow rates.

### Illustrated Embodiments of Infusion Apparatus

Referring to FIG. 1, an infusion apparatus 100 according to one embodiment includes a syringe module 200 and an actuator module or a pump module 300. The syringe module 200 can contain infusion fluid, and includes a syringe barrel 210 and a piston 250 inserted in the syringe barrel 210. The piston 250 can move to pressurize the infusion medicine for infusion. The actuator module 300 is coupled to the syringe barrel 210, and includes compression springs for pushing the piston 250 using spring force.

### Syringe Barrel

In embodiments, the syringe barrel 210 may be made of a transparent or translucent plastic material for showing the infusion fluid. The syringe barrel 210 may include scale markings on its wall for measuring the amount of the infusion fluid contained therein. The syringe barrel 210 includes a nozzle 212. A flexible infusion tube may be connected to the nozzle 212.

### Shape of Syringe Barrel

In embodiments, the syringe barrel 210 has a generally rectangular sectional shape with four rounded corners, as shown in FIGS. 3 and 9. But, the invention is not limited thereto. The sectional shape of the syringe barrel 210 may be, for example, square or oval.

### Piston

The piston 250 includes a piston body 252 and seals 254 covering the piston body 252. The seals 254 contact the sidewalls of the syringe barrel 210 for sealing between the piston and the sidewalls.

### Actuator Module

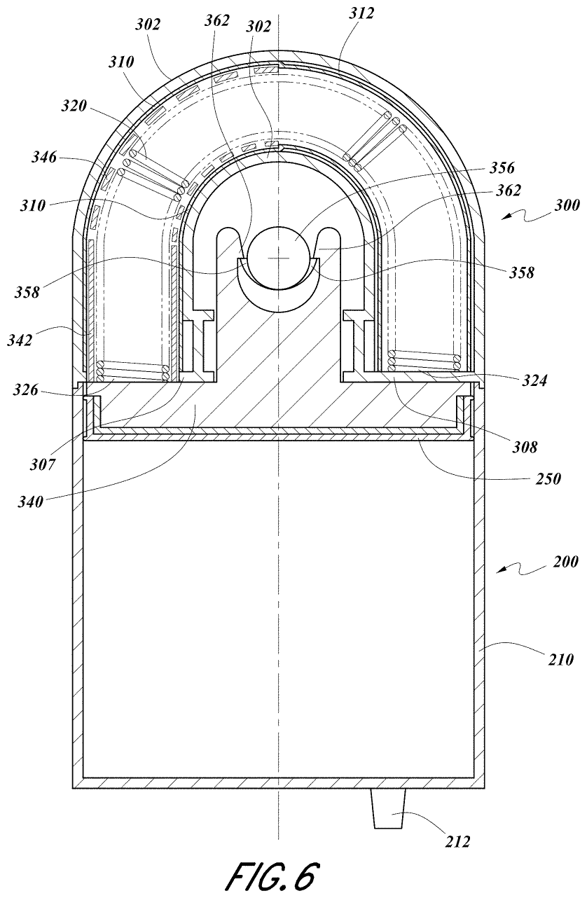

Referring to FIGS. 5-8, the actuator module 300 includes a housing 302 and two U-shape spring guide tubes 310 and 312 that are accommodated and fixed in the housing 302. The actuator module 300 further includes two compression springs 320 and 322 that are inserted in the guide tubes 310 and 312, respectively. The actuator module 300 further includes a pressure plate 340 that receives spring force from the springs 320 and 322. The pressure plate 340 is coupled to the piston 250 and transmits the spring force to the piston 250. The actuator module 300 also includes a locking mechanism that locks or unlocks the movement of the pressure plate 340 of the actuator module 300.

### Housing

The housing 302 includes a front member 304 and a rear member 306 that are coupled to each other and define a space for accommodating the tubes 310 and 312. Further, to provide secure engagement of the actuator module 300 and the syringe module 200, the housing 302 has a coupling structure that can engage with a counterpart coupling structure of the syringe barrel 210. The secure engagement is maintained during infusion of the infusion fluid.

### Two U-Shape Spring Guide Tubes

The U-shape spring guide tubes 310 and 312 are arranged side by side in the housing 302 and fixed to the housing 302. Each of the guide tubes 310 and 312 includes a first end 314 and a second end 316 and a rounded channel portion 318 that interconnects the first end 314 and the second end 316. Each of the spring guide tubes 310 and 312 further includes a linear channel portion between the first end 314 and the rounded channel portion 318, and another linear channel portion between the second end 316 and the rounded channel portion 318.

### Bottom Walls of Housing Blocking Ends of U-Shape Tubes

In embodiments, the housing 302 includes outer walls, inner walls and bottom walls 307 and 308. The outer walls, inner walls and bottom walls define a space for receiving the tubes 310 and 312. In one embodiment illustrated in FIG. 6, the bottom wall 308 closes or blocks the second end 316 of the tube 310. The bottom wall 307 includes a hole that communicates with the first end 314 of the tube 310. Similarly, the bottom wall 307 closes or blocks the second end 316 of the tube 312. The bottom wall 308 includes a hole that communicates with the first end 314 of the tube 312.

### Locations of Ends of Guide Tube

Referring to FIGS. 5-8, the second end 316 of the guide tube 310 is closed or blocked by the bottom wall 308 and located at the right side of the actuator 312, and the first end 314 of the guide tube 310 is open through the hole of the bottom wall 307 and located at the left side of the actuator 312. Contrastingly, the second end 316 of the tube 312 is closed or blocked by the bottom wall 307 and located at the left side of the actuator module 300, and the first end 314 of the tube 312 is open through the hole of the bottom wall 308 and located at the right side of the actuator module 300.

### Two Compression Springs

Each of the compression springs 320 and 322 are inserted in the corresponding one of the guide tubes 310 and 312. In the guide tubes 310 and 312, the springs 320 and 322 are bent to have a U-shape or J-shape. In embodiments, the springs 320 and 322 are at least partly accommodated in the tube 310 and 312. In one embodiment, substantially the entire portion of each of the springs 320 and 322 is accommodated in the corresponding tube in the fully compressed state. When each of the springs 320 and 322 expands, one end of each spring moves out of the corresponding tube and pushes the pressure plate 320 for infusion.

### Shape of Springs

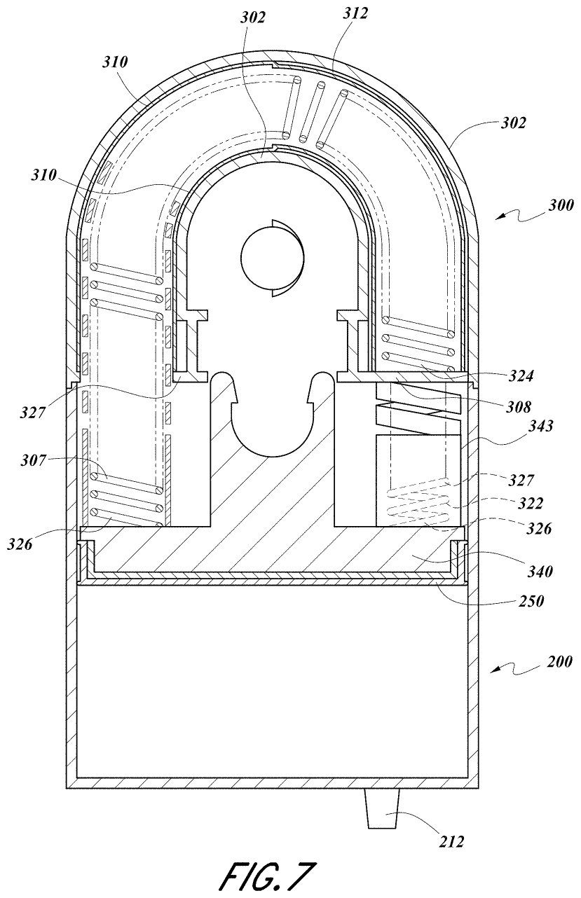

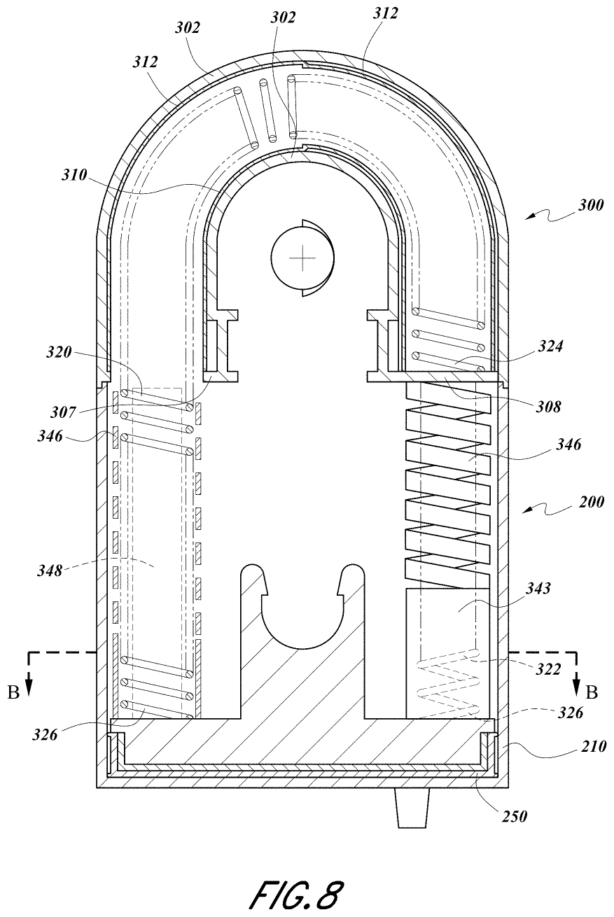

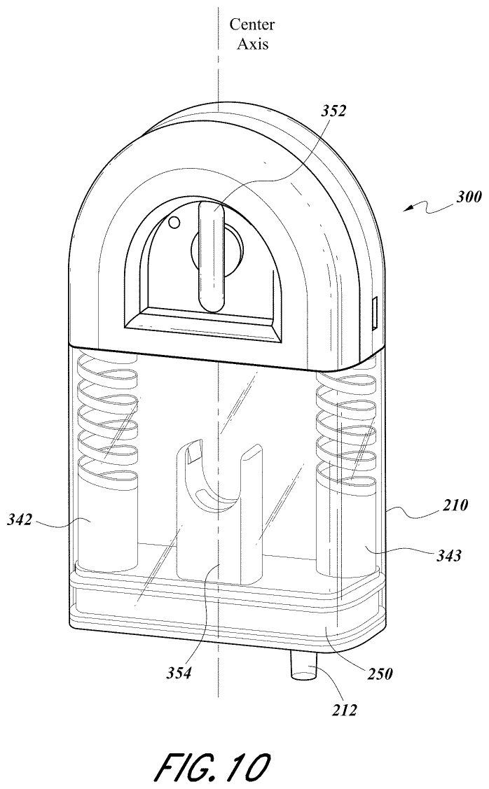

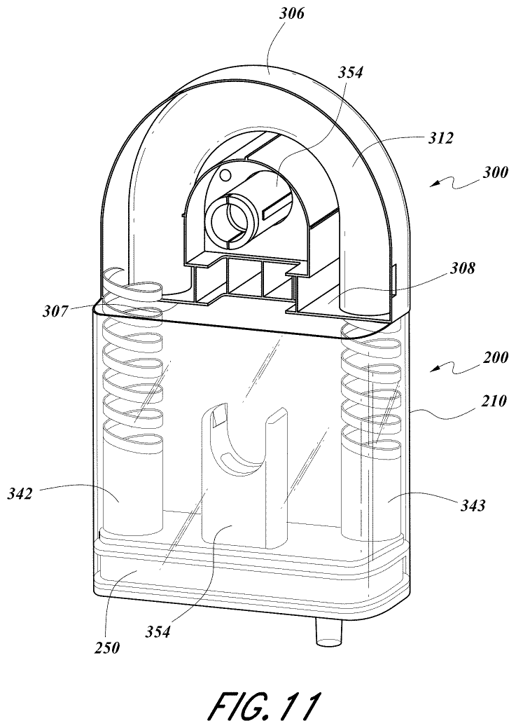

In one embodiment shown in FIG. 6, in fully compressed state, each spring 320 or 322 is accommodated in the corresponding guide tube and forms a U-shape. In an expanded state, as shown in FIGS. 7 and 8, a portion of each spring is still bent and accommodated in the corresponding tube, while the rest of the spring is located outside the tube and extends linearly such that the spring forms a J-shape. In one embodiment, each spring is formed of a single wire which is helically coiled.

### Fixed End and Movable End of Each Spring

As shown in FIGS. 6-8, the spring 320 includes a stationary end or fixed end 324 that contacts and is supported by the bottom wall 308 located on the right side of the actuator and a movable end 326 that can move through the hole formed in the bottom wall 307 located at the left side of the actuator 300. When the spring 320 expands, the movable end 326 moves linearly along the center axis and pushes the pressure plate 340. The fixed end 324 of the spring 320 pushes the bottom wall 308, but does not move as the bottom wall 308 functions as a stopper or spring support. In one embodiment, the fixed end 324 may be firmly secured to the bottom wall 308 or may be placed over the bottom wall 308 without firm attachment to the bottom wall 308. Similarly, the spring 322 includes a stationary end or fixed end 324 that contacts and is supported by the bottom wall 307 located on the left side of the actuator and a movable end 326 that can move through the hole formed in the bottom wall 308 located at the right side of the actuator 300. When the spring 322 expands, the movable end 326 moves linearly along the center axis and pushes the pressure plate 340. The fixed end 324 of the spring 322 pushes the bottom wall 307, but does not move as the bottom wall 307 functions as a stopper or spring support. In one embodiment, the fixed end 324 of the spring 322 may be firmly secured to the bottom wall 307 or may be placed over the bottom wall 307 without firm attachment to the bottom wall 307.

### Each Spring's Fixed End and Movable End Locating at Opposite Sides

In the illustrated embodiment, when the spring 320 is received in the tube 310, the fixed end 324 is locate at the right side of the actuator module 300, and the movable end 326 of the spring 320 is located at the left side of the actuator module 300. When the spring 322 is received in the tube 312, the fixed end 324 of the spring 322 is locate at the left side of the actuator module 300 and the movable end 326 of the spring 322 is located at the right side of the actuator module 300.

### Both Movable End and Fixed End Facing Piston

In embodiments, each of the spring 320 and 322 is bent in the corresponding guide tube 310 or 312 such that each spring includes a bent portion between two ends 324 and 326. In the embodiment illustrated in FIGS. 6-8, both the fixed end 324 and the movable end 326 face the piston 250. The force at the movable end 326 of the spring 320 is applied to the piston 250 while the force at the fixed end 324 of the spring 320 is applied to the corresponding bottom wall 308. The force at the movable end 326 of the spring 322 is applied to the piston 250 while the force at the fixed end 324 of the spring 322 is applied to the corresponding bottom wall 307. In the illustrated embodiment, the supporting walls 307 and 308 are generally perpendicular to the center axis, but not limited thereto. The locations and angles of the supporting walls can vary and may cause the fixed ends of the springs to face in another direction which forms an acute angle with the center axis.

### Movable Ends of Two Springs Locating Diagonally

In one embodiment shown in FIG. 9, the movable end 326 of the spring 320 pushes at a left-front corner 345 of the pressure plate 340 whereas the movable end 326 of the spring 322 pushes at a right-rear corner 347 of the pressure plate 340. The movable ends 326 are arranged diagonally with respect to the center axis of the apparatus 100. The movable end 326 of the spring 320 is distanced from the center axis of the apparatus 100 by a distance which is substantially the same as a distance by which the movable end 326 of the spring 322 is distanced from the center axis of the apparatus 100. The above configuration provides balanced application of the spring forces.

### Size of Springs

The bent spring 320 has a width that is a dimension between the movable end and the fixed end measured in a width direction of the infusion apparatus and a thickness that is a dimension measured in a thickness direction of the infusion apparatus. In embodiments, the width of the spring 320 is smaller than that of the syringe barrel, and the thickness of the spring 320 is smaller than a half the thickness of syringe barrel. The bent spring 322 has dimensions substantially the same as those of the spring 320.

### Size of Bent Shape Springs in Width Direction of Infusion Apparatus

In embodiments, the bent springs 320 and 322 have a dimension in the width direction smaller than the width of the syringe barrel. In one embodiment, the bent springs 320 and 322 can be arranged side by side and located closely to each other such that both the bent springs 320 and 322 can be accommodated or fit through the rectangular sectional shape of the actuator when viewed from the top of the actuator along the center axis. In another embodiment, in a top view, both the springs 320 and 322 can be accommodated or fit through a rectangular sectional shape of the piston. Thus, the entire portion of the springs 320 and 322 can overlap the piston 250 in a viewing direction along the center axis. But, the invention is not limited thereto. In another embodiment, if it is allowed for the actuator module to have a size bigger than that of the syringe barrel, the spring may have a portion that does not overlap the piston in a top view.

### Buckling

When expanding, a portion of each of the springs 320 and 322 comes out of the corresponding one of the guide tubes 310 and 312 and moves along the center axis in the syringe barrel 210. This portion of each spring may be subject to buckling during expansion. Thus, the actuator module 300 further includes structures for preventing the springs 320 and 322 from buckling.

### Buckling-Prevention Structure

In embodiments, the actuator module 300 includes two buckling-prevention guides 342 and 343. In one embodiment, the guide 342 and 343 are fixed to the pressure plate 340. Each of the guides 342 or 343 extends upwardly from the pressure plate 340 for receiving the movable end 326 of the corresponding spring 320 or 322. Each guide 342 or 343 includes a rigid pipe portion 344 and a bendable portion 346 extending from the rigid portion 344. The bendable spring portion 346 can be bent in conformation with the rounded or curved portion of the corresponding tube 310 or 312 as shown in FIG. 6. In one embodiment, each of the rigid portion and the bendable portion 346 has a bending stiffness greater than that of the springs 320 and 322 and sufficient to prevent the spring 320 or 322 from buckling when the bendable portion is out of the tube as shown in FIG. 8. In the illustrated embodiment, the bendable portion 346 has a spring-like shape. In one embodiment, the buckling-prevention guide may be made of a material the same as that of the pressure plate.

### Length of Buckling-Prevention Guide

In one embodiment, the buckling-prevention guide has a length the same as or slightly greater than a distance between the pressure plate 340 and the first end of the tube 310 or 312. The distance is measured when the pressure plate 340 is located at its lowest position. In this embodiment, a distal end portion of the buckling-prevention guide is located in and supported by the corresponding guide tube. This configuration provides more effective buckling-prevention function as the proximal end is fixed to the pressure plate 340 and the distal end is restricted by the corresponding guide tube. However, the invention is not limited thereto. In another embodiment, the length of the buckling-prevention guide may be slightly smaller than the distance between the pressure plate 340 and the first end of the tube.

### Diameter of Buckling-Prevention Guides Relative to U-Shape Guide Tubes

Each buckling-prevention guide 342 or 343 has an inner diameter slightly greater than an outer diameter of the spring 320 or 322, and the buckling-prevention guide 342 or 343 has an outer diameter slightly smaller than an inner diameter of the tube 310 or 312. Thus, the tubes 310 and 312 can receive the guides 342 and 343, respectively, and the springs 320 and 322 can be at least partly retained in the guides 342 and 343, respectively. In the illustrated embodiment, each tube includes a portion having an enlarged inner diameter for receiving the corresponding buckling-prevention guide.

### Another Example of Buckling-Prevention Structure

In another embodiment, the buckling-prevention structure is a pole 348 that extends from the pressure plate and is inserted in the spring as shown in FIG. 8. The pole 348 may be a pipe or a rod. The pole 348 is deformable and deforms in conformation with the rounded portion of the corresponding U-shape guide tube when the spring 320 or 322 is received in the corresponding tube 310 or 312 in the compressed state. However, the pole 348 has a bending stiffness greater than that of the springs and sufficient to prevent the spring 310 or 312 from buckling when the pole 348 is out of the tube as shown in FIG. 8. In one embodiment, to prevent the spring from buckling, the actuator module 300 may include both the spring-receiving guides 342 and 343 and the inserting poles 348.

### Telescopic Buckling-Prevention Structure

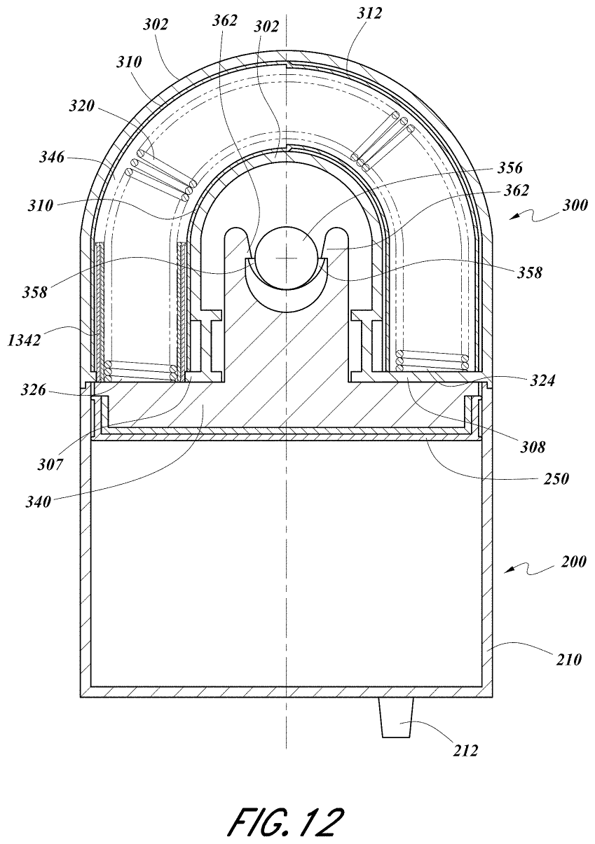

In one embodiment illustrated in FIGS. 12-14, the actuator module 300 includes two telescopic buckling-prevention structures 1342 and 1343 that may be fixed to the pressure plate 340. The telescopic pipe structure 1342 includes two or more pipes that can expands and collapses. In a collapsed state, the telescopic pipe structure 1342 can be received in the linear channel portion of the U-shape tube 310, and there is no need to bend any portion of the telescopic pipe structure to be received in the U-shape tube 310. Thus, each pipe of the telescopic pipe structure can have a bending stiffness greater than that of the bendable portion 346 shown in FIG. 6. Further, the telescopic pipe structure 1342 has a bending stiffness greater than that of the springs 320 and 322 and sufficient to prevent the spring 320 from buckling when the telescopic pipe structure expands as shown in FIG. 14. The telescopic pipe structure 1343 can have the same structure and function as those of the telescopic pipe structure 1342, while the telescopic pipe structure 1343 receives the spring 322 and is received in the U-shape tube 312.

### Telescopic Pipes

Each of the telescopic pipe structures 1342 and 1343 includes an inner pipe 1344 and an outer pipe 1346 receiving the inner pipe 1344. In one embodiment illustrated in FIGS. 12-14, the inner pipe 1344 includes a bottom end fixed to the pressure plate 340. When the spring 320 or 322 pushes the pressure plate 340, the inner pipe 1344 moves relative to the outer pipe 1346 along the center axis until the outward flange of the inner pipe 1344 contacts the inward flange of the outer pipe 1346. Then, the inner pipe 1344 and the outer pipe 1346 move together along the center axis until the piston reaches the bottom wall of the syringe barrel 210. In the illustrated embodiment, the outer pipe 1346 further includes an outward flange at the top thereof and the U-shape tube 310 or 312 includes an inward flange cooperating with the outward flange of the outer pipe 1346 to inhibit the outer pipe from being separated from the U-shape tube.

### Locking Mechanism

The springs 320 and 322 in the actuator module 300 tend to expand when there is no restriction. Thus, the actuator module 300 includes a locking mechanism for maintaining the compressed state of the springs 320 and 322. The locking mechanism includes a locking knob 352 coupled to the housing 310 and a locking post 354 attached to the pressure plate 340 and capable of engaging with the locking knob 352. The locking knob 352 can rotate about a knob axis perpendicular to the center axis. The knob 352 includes a cylindrical body 356 that includes protrusions 358 diametrically arranged on outer surface of the body 356. The locking post 354 includes a recess 360 for receiving the cylindrical body 356 and mating protrusions 362 formed on opposing surfaces of the recess 360. The protrusions 358 of the knob and the protrusions 362 of the locking post 354 can engage with each other by snap-fitting. FIG. 6 shows the engagement of the protrusions 356 and 358. When rotating the knob 352, the engagement is released, which allows the springs 320 and 322 to expand as shown in FIGS. 7 and 8.

### Locked State

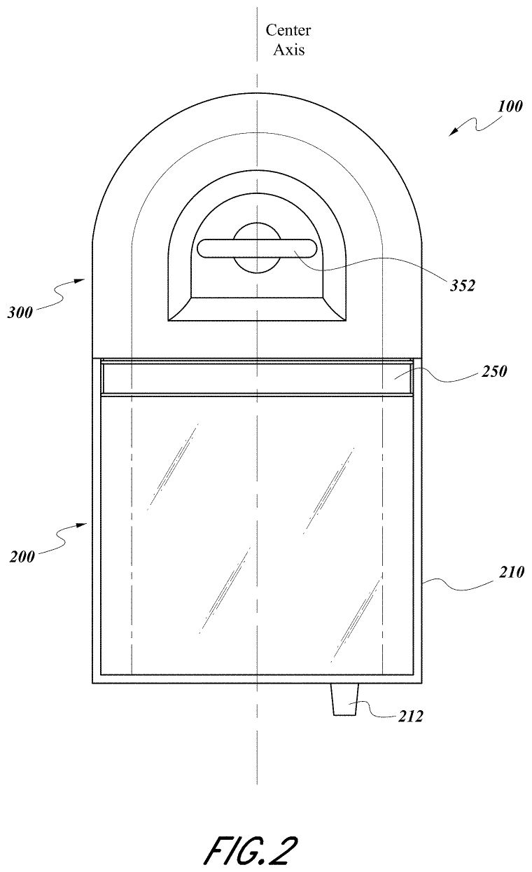

Referring to FIGS. 1, 2 and 6, the springs 320 and 322 are compressed and accommodated in the tubes 310 and 312, respectively. The springs 320 and 322 may be fully compressed. The locking mechanism inhibits the pressure plate 340 from moving although the compressed springs 320 and 322 apply force to the pressure plate 340. In this locked state according to one embodiment, the substantially entire portion of each of the springs 320 and 322 is re retained in the corresponding guide tube 310 or 312, whereas the movable end may be located slightly outside the tube in the locked state in another embodiment. The buckling-prevention guides 342 and 343 are also retained in the guide tubes 310 and 312, in which the deformable portions 346 are bent in conformation with the rounded portions 318 of the corresponding tubes 310 and 312. In embodiments, in the locked state, each of the springs 320 and 322 has a U-shape.

### Infusion

In embodiments, when rotating the knob 352, the engagement of the knob 352 and the locking post 354 is released, and then, by the force of the springs 320 and 322, the piston 250 and the pressure plate 330 start moving. As shown in FIG. 7, the movable ends 326 of the springs 320 and 322 come out of the guide tubes 310 and 320. Further, the rigid portions 344 of the buckling-prevention guides 342 and 343 come out of the guide tubes 310 and 320 along with the springs 310 and 312. Thus, the buckling-prevention guides 342 and 343 prevent the springs 320 and 322 from buckling during the expansion of the springs 320 and 322.

### Linear Movement of Movable Ends

When the springs 320 and 322 expand, at least a portion of each spring that is bent and located in the tube comes out of the corresponding tube and extends linearly. Therefore, the compression springs 320 and 322 are transformed from the U-shape to a J-shape during expansion. Further, the compression springs 320 and 322 push linearly the pressure plate 340 at the opposite sides of the piston 250, respectively, as the movable ends 326 of the springs 320 and 322 move linearly.

### Completion of Infusion

FIG. 8 shows the completion of infusion when the piston 250 stops by the bottom of the syringe barrel 210. In this state according to one embodiment, the springs 320 and 322 expand to have a length smaller than their free length.

### Examples of Spring's Dimensions

In one embodiment, ratio of free length of the helical spring to the length of syringe barrel (which is a travel distance of the piston) is greater than about 6:1. Ratio of free length to the solid length of the helical spring is greater than about 4:1. With these ratios, force decrease of the spring as displacement decreases at the beginning of travel (beginning of infusion) versus at the end of travel (end of infusion) should be less than 20%. The greater the ratio of free length versus the piston travel length is, the smaller the ratio of force decrease.

### Comparison Between Bent Spring and Unbent Spring

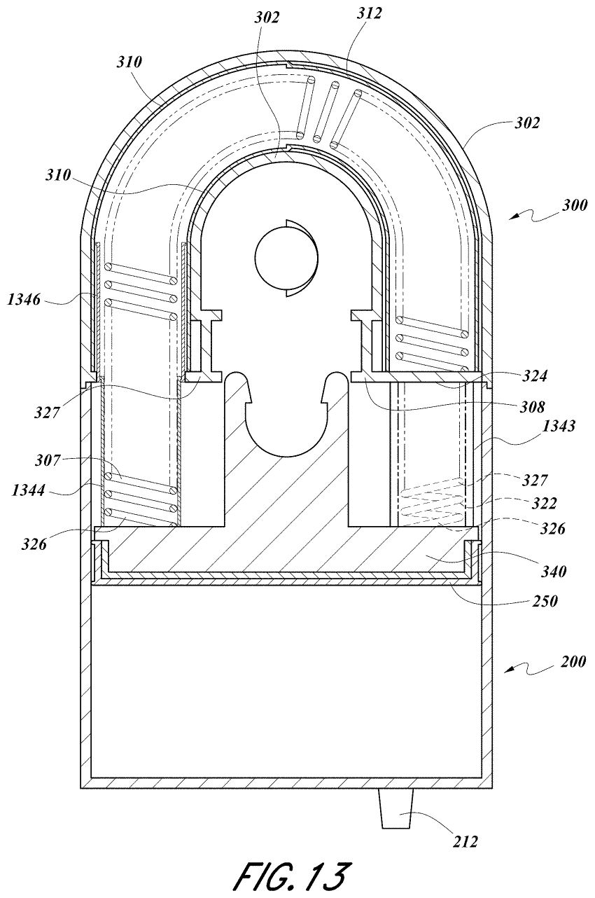

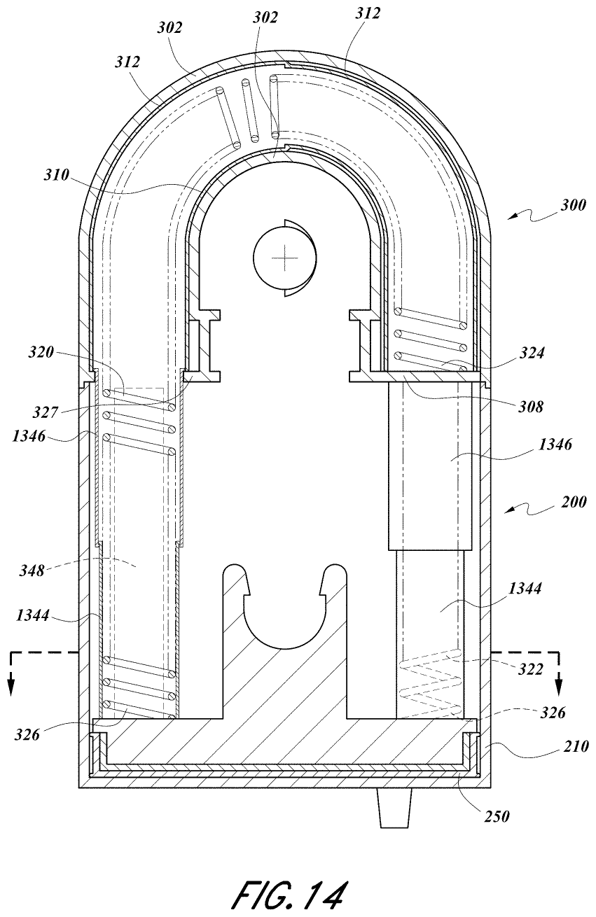

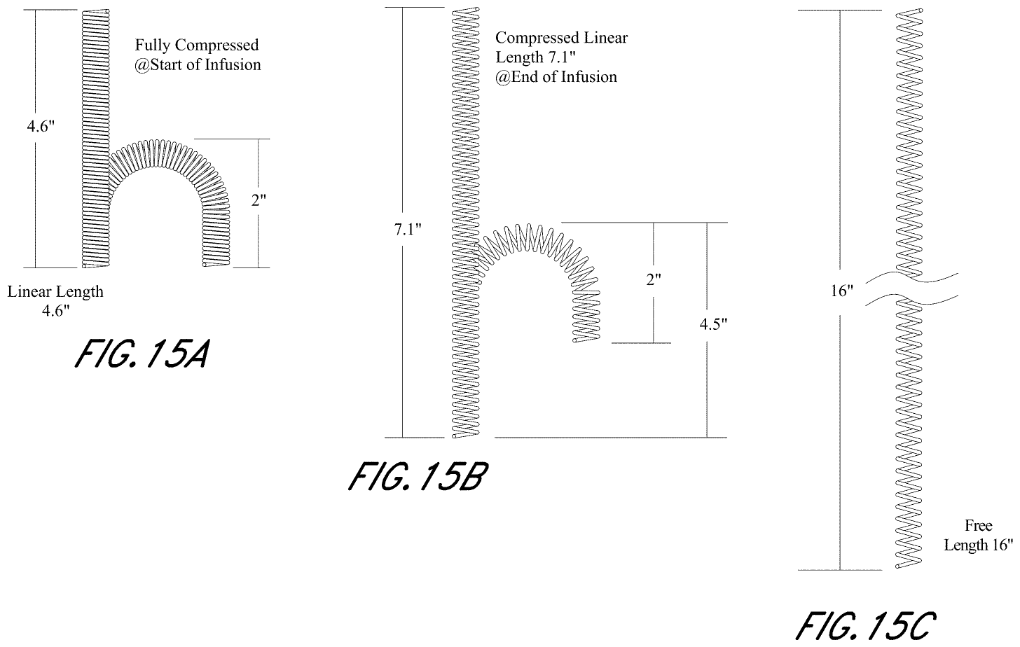

In embodiments, to avoid the size increase, springs are bent in a U-shape or J-shape. FIG. 15A shows the spring in its unbent, compressed state and bent, compressed state shown in FIGS. 1 and 6. In one embodiment, the spring has a height of 2 inch in its bent, compressed state while the spring has a height of 4.6 inch in its unbent, compressed state. FIG. 15B shows the spring in its unbent, expanded state and its bent, expanded state shown in FIG. 8. In one embodiment, the spring has a height of 4.5 inch in its bent, expanded state while the spring has a height of 7.1 inch in its unbent, expanded state. FIG. 15C shows the spring which is unbent and free of exterior force, in which the spring has a height of 16 inch. It is notable that when the springs 320 are 322 are used without bending, the height of the infusion apparatus is much greater than when the springs are used in their bent state.

### Width and Thickness of Apparatus

In embodiments, the springs 320 and 322 do not have a portion that is located outside sidewalls of the syringe barrel 210 when viewing the springs 320 and 322 along the center axis. In this example, outer dimensions of syringe barrel 210 may define the width and the thickness of the apparatus.

### Examples of Spring Dimensions and Properties

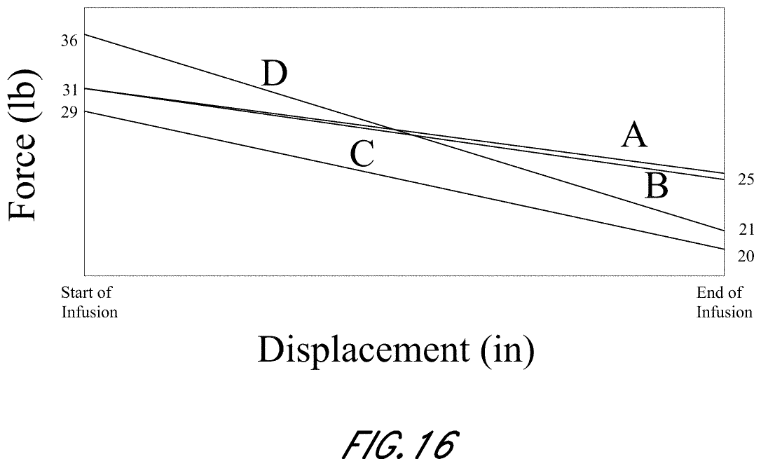

FIG. 16 shows various examples of changes in spring force of various springs listed in

TABLE 1

| | Free | Spring | Force (lb) at | Force (lb) at | Force |
|---|---|---|---|---|---|
| | Length | Constant | Start of Infusion | End of Infusion | Decrease |
| | (in) | (lb/in) | (Pressure, psi) | (Pressure, psi) | (%) |
| A | 18 | 1.1 | 30.8 (10.3) | 25.3 (8.4) | −18 |
| B | 16 | 1.3 | 31.2 (10.4) | 24.7 (8.2) | −21 |
| C | 12 | 1.8 | 28.8 (9.6) | 19.8 (6.6) | −31 |
| D | 10 | 3 | 36 (12) | 21 (7) | −42 |

### Flow Rate of Apparatus

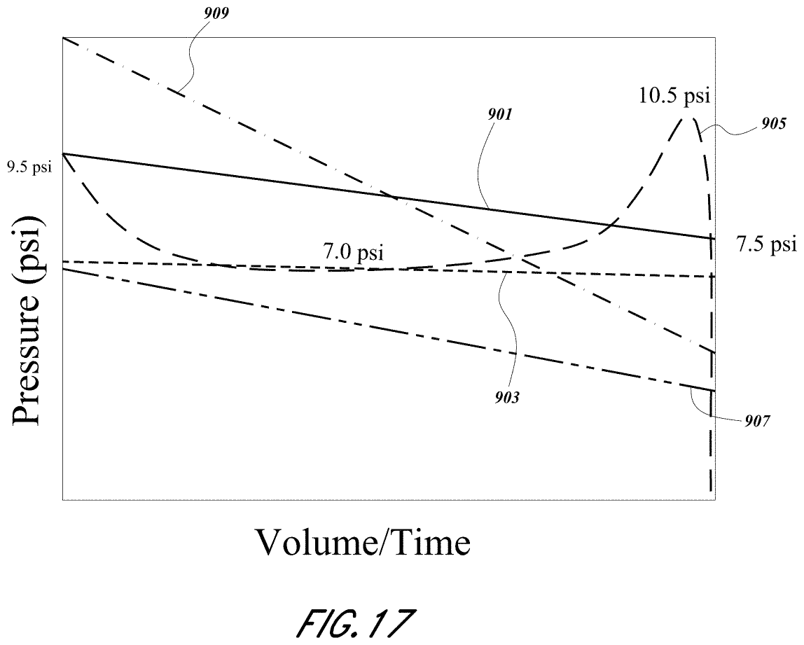

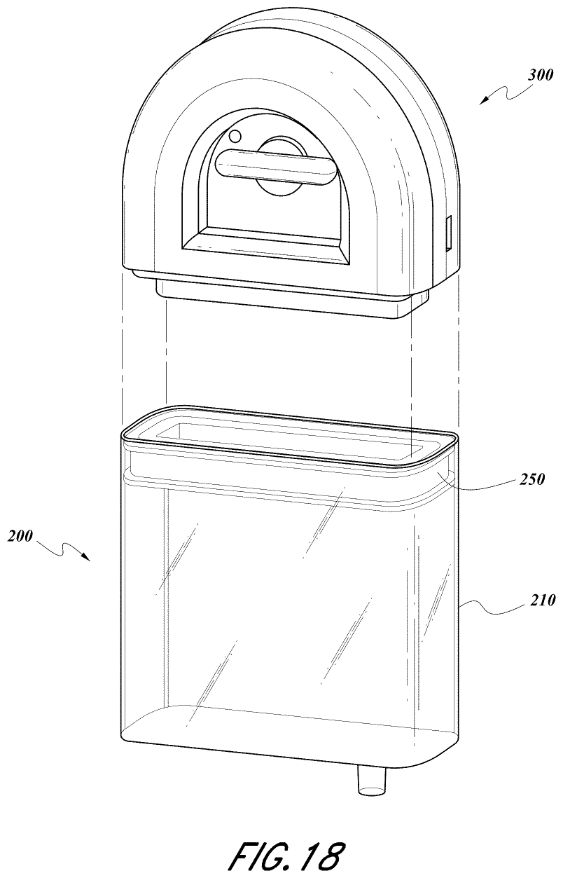

The apparatus 100 according to embodiments provides better flow rate accuracy, i.e., least pressure (flow rate) variation from beginning of infusion to end of infusion. When combined with a pressure regulator, flow rate accuracy is even competitive with electronic pumps. Referring to FIG. 17, line 901 shows the pressure change of the apparatus without a regulator, and line 903 shows the pressure change of the apparatus with a regulator. Comparing with line 905 showing the pressure changes of an elastomeric bladder type pump and lines 907 and 909 showing the pressure changes of other spring powered pumps, the infusion apparatus 100 according to embodiments of the invention is significantly advantageous in the aspect of the flow rate accuracy.

### After Completion of Infusion

After completion of infusion, the syringe module 200 is separated from the actuator module 300. The separated syringe module 200 is discarded, and the actuator module 300 may be reused. For reusing the actuator module 300, the knob 352 is rotated in its locking state. The pressure plate 340 is pushed back to compress the springs 320 and 322. In the embodiment illustrated in FIGS. 6-8, each buckling-prevention structure 342 or 343 is received in the corresponding tube 310 or 312. In the embodiment illustrated in FIGS. 12-14, each buckling-prevention structure 1342 or 1343 is received in the corresponding tube 310 or 312. When the springs 320 and 322 are fully compressed, the locking post 354 and the knob 352 are engaged with each other by snap fit. In another embodiment, both the actuator module 300 and the syringe module 200 are discarded after completion of infusion without reusing.

### Supplying Actuator Module and Syringe Module

In one embodiment, a patient user can receive the infusion apparatus 100 in its assembled state from a supplier, for example, a pharmaceutical company, a health care provider or a pharmacist. In the assembled infusion apparatus 100, the actuator module 300 is engaged with the syringe module 200 that is filled with infusion fluid. After completion of infusion, a user can separate the syringe module 200 from the actuator module 300, and disposed the syringe module 200 while keeping the actuator module 300 for reuse.

### Supplying New Syringe Module

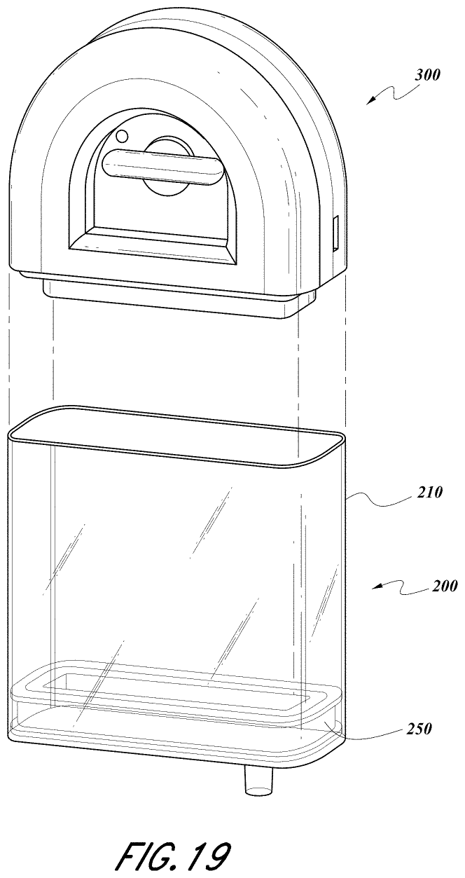

In embodiments, a new syringe module 200 can be supplied to a patient user that uses a used actuator module from a medicine supplier, for example, a health care provider or a pharmacist. The user receives the syringe module 200 that is filled with infusion medicine. As shown in FIG. 18, the piston is located at the top of the syringe barrel 210. In one embodiment, the top of the syringe module 200 may be sealed with a cover sheet. A patient user takes off the cover sheet and can assemble the syringe module 200 with the used or new actuator module 300 for infusion.

Filling Syringe Module with Infusion Fluid

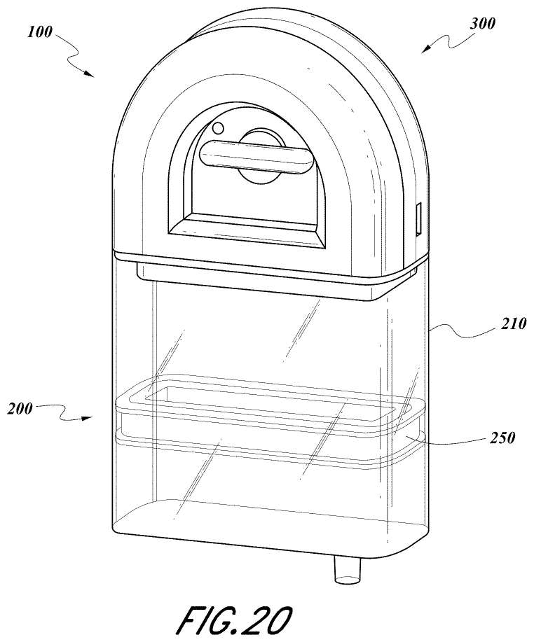

In another embodiment, an empty syringe module 200 is first engaged with the actuator module 300 as shown in FIG. 19. And then, infusion fluid is supplied into the syringe barrel 210 through the nozzle as shown in FIG. 20. In the illustrated embodiment, the springs are compressed and restricted by the locking mechanism and does not resist movement of piston toward the actuator. Thus, almost no pressure is required to fill the syringe barrel.

### Advantageous Effects

As discussed above, the configuration of the infusion apparatus according to embodiments of the invention provides the following advantages:
Small size which is competitive with elastomeric bladder type infusion pumps, for example, the size for 100 mL infusion apparatus as small as 1.2″ (thickness)×2.5″ (width)×4″ (height), which is approximately the size of a cigarette box;
improved flow rate accuracy, i.e. least pressure (flow rate) variation from beginning to end, which is far superior to elastomeric pump and other currently existing spring type pumps;
reusable actuator;
pre-fillable syringe barrel; and
easy filling of medicine fluid.

Although embodiments are described as above, it should be understood that numerous and various modifications can be made without departing from the spirit of the invention. Accordingly, the invention is limited only by the following claims.

What is claimed is:

1. An infusion apparatus comprising:
a syringe module configured to contain infusion medicine, wherein the syringe module comprises a syringe barrel and a piston, wherein the syringe barrel comprises a bottom and side walls extending from the bottom in a direction, wherein the syringe barrel further comprises an outlet for discharging infusion medicine, wherein the piston is disposed in the syringe barrel and movable in the direction, wherein the piston is configured to push infusion medicine contained in the syringe barrel toward the outlet; and
an actuator module configured to engage with the syringe module, wherein the actuator module is further configured to push the piston in the direction, wherein the actuator module comprises a housing, two or more spring guide channels arranged side by side in the housing and two or more compression springs, each of which is inserted in the corresponding spring guide channel,
wherein each spring guide channel comprises a first end, a second end and a rounded portion between the first and second ends,
wherein each spring is compressible and configured to expand between a compressed state and an expanded state, wherein each spring comprises a fixed end, a movable end and a bent portion in conformation with the rounded portion of the corresponding spring guide channel, wherein the actuator comprises spring supports, each of which configured to block the second end of the corresponding spring guide channel such that the fixed end contacts and is stopped by the corresponding spring support, wherein the movable end is configured to move in the direction and further configured to push the piston when the spring expands, which causes the piston to move in the direction,
wherein each spring guide channel is configured to retain the corresponding spring therein such that the fixed end, the movable end and the rounded portion of the corresponding spring overlap the piston in the compressed state and the expanded state when viewed in the direction while the fixed end and the movable end are located at opposite sides of the infusion apparatus,
wherein the movable end of a first one of the springs and the movable end of a second one of the springs are located at opposite sides of the infusion apparatus when viewed in the direction.

2. The infusion apparatus of claim 1, wherein the entire portion of each spring overlaps the piston when viewed in the direction.

3. The infusion apparatus of claim 1, wherein the piston has a generally rectangular shape when viewed in the direction, wherein the movable end of the first spring is configured to push the piston at a first corner of the piston, and the movable end of the second spring is configured to push the piston at a second corner of the piston that is diagonally opposite to the first corner.

4. The infusion apparatus of claim 1, wherein each spring has a U-shape in the compressed state, and a J-shape in the expanded state and while the movable end is moving in the direction.

5. The infusion apparatus of claim 1, wherein the movable end of each spring is located in the housing of the actuator module in the compressed state and located in the syringe barrel in the expanded state while the fixed end is located in the housing of the actuator in both the compressed state and the expanded state.

6. The infusion apparatus of claim 1, wherein both the movable end and the fixed end of each spring face the piston in the compressed state and in the expanded state.

7. The infusion apparatus of claim 1, wherein, when the springs expand, the bent portion of each spring in the compressed state is partly deformed to extend linearly in the expanded state.

8. The infusion apparatus of claim 1, wherein each spring is formed of a single wire that is helically coiled and interconnects the fixed end and the movable end.

9. The infusion apparatus of claim 1, wherein each spring guide channel further comprises a first straight extension connected to the first end and a second straight extension connected to the second end, and the rounded portion of each channel interconnects the first and second straight extensions.

10. The infusion apparatus of claim 8, wherein the rounded portion is semi-circular or arcuate.

11. The infusion apparatus of claim 1, wherein the actuator module further comprises a locking mechanism configured to maintain the springs in the compressed state.

12. The infusion apparatus of claim 1, wherein in the expanded state of the springs, the piston contacts the bottom of the syringe barrel.

13. The infusion apparatus of claim 1, wherein further comprising a pressure plate that contacts the movable end of each spring and is coupled to the piston to push the piston when the springs expand.

14. The infusion apparatus of claim 12, wherein the actuator module further comprises two or more buckling-prevention guides extending from the pressure plate, wherein the movable end of each spring is inserted in the corresponding buckling-prevention guide that has a bending stiffness greater than that of the corresponding spring.

15. The infusion apparatus of claim 13, wherein each buckling-prevention guide comprises an end portion that is deformable and configured to deform in conformation with the rounded portion of the corresponding spring guide channel when the buckling-prevention guide is inserted in the corresponding spring guide channel.

16. The infusion apparatus of claim 12, wherein each buckling-prevention guide comprises telescopic pipes that receive the movable end of the spring, wherein each of the telescopic pipes is movable with respect to the rest of the telescopic pipes when the spring moves the piston in the direction.

17. A method of infusing medicine to a patient, the method comprising:
attaching a syringe module to an actuator module to form an infusion apparatus, wherein the infusion apparatus comprises:
the syringe module containing infusion medicine therein, wherein the syringe module comprises a syringe barrel and a piston, wherein the syringe barrel comprises a bottom and side walls extending from the bottom in a direction, wherein the syringe barrel further comprises an outlet for discharging medicine, wherein the piston is disposed in the syringe barrel and movable in the direction, wherein the piston is configured to push infusion medicine contained in the syringe barrel toward the outlet,
the actuator module configured to push the piston in the direction when the actuator is engaged with the syringe module, wherein the actuator module comprises a housing, two or more spring guide channels arranged side by side in the housing and two or more compression springs, each of which is retained by the corresponding spring guide channel, wherein each spring guide channel comprises an first end, a second end and a rounded portion between the first end and the second end, wherein each spring is compressible and configured to expand between a compressed state and an expanded state, wherein each spring comprises a fixed end, a movable end and a bent portion in conformation with the rounded portion of the corresponding guide channel, wherein the actuator comprises spring supports, each of which configured to block the second end of the corresponding spring guide channel such that the fixed end contacts and is stopped by the corresponding spring support, wherein the movable end is configured to move in the direction and further configured to push the piston when the spring expands, which causes the piston to move in the direction;
wherein each spring guide channel is configured to retain the corresponding spring such that the fixed end, the movable end and the rounded portion of the corresponding spring overlap the piston when viewed in the direction, wherein the fixed end and the movable end are located at opposite sides of the infusion apparatus, wherein the movable end of a first one of the springs and the movable end of a second one of the springs are located at opposite sides of the infusion apparatus when viewed in the direction;
releasing the springs of the actuator module from their compressed state such that the springs expand and the movable ends of the springs push the piston, which causes the piston to move in the direction for discharging the infusion medicine through the outlet;
upon completion of infusion, detaching the syringe module from the actuator module; and
retracting the springs of the actuator module to the compressed state.

18. The method of claim 17, further comprising engaging another syringe module containing infusion medicine with the actuator module for infusion.

19. The method of claim 17, wherein the syringe module is filled with medicine before attaching the syringe module to the actuator module.

20. The method of claim 17, wherein each spring has a U-shape in the compressed state, and becomes a J-shape during the infusion.
